(19) United States
(12) Patent Application Publication
(10) Pub. No.: US 2008/0132441 A1
(43) Pub. Date: Jun. 5, 2008

(54) Methods and compositions for modulating medial-lateral neuronal topographic maps

(76) Inventor: Yimin Zou, San Diego, CA (US)

Correspondence Address: MICHAEL BEST & FRIEDRICH LLP, ONE SOUTH PINCKNEY STREET, P O BOX 1806, MADISON, WI, 53701, US

(21) Appl. No.: 11/904,594

(22) Filed: Sep. 27, 2007

Related U.S. Application Data
(63) Continuation of application No. PCT/US06/11711, filed on Mar. 31, 2006.
(60) Provisional application No. 60/666,989, filed on Mar. 31, 2005.

(51) Int. Cl. A61K 38/00; A61P 25/00

(52) U.S. Cl. 514/2

(57) ABSTRACT

Methods and kits are provided for modulating the axonal growth of a neuron comprising contacting the neuron with a Wnt3 polypeptide or expressing an exogenous polynucleotide encoding a Wnt3 polypeptide. Also provided are compositions for modulating the medial-lateral axonal growth of a neuron comprising a Wnt3 polypeptide and a pharmaceutically acceptable carrier or diluent and methods for administering such compositions to subjects.

### CROSS-REFERENCE TO RELATED APPLICATIONS

This application is a continuation under 35 U.S.C. 1.111 of International Application No. PCT/US2006/011711, filed Mar. 31, 2006, which claims priority to U.S. provisional application 60/666,989, filed Mar. 31, 2005, which are incorporated herein by reference in their entirety.

### STATEMENT REGARDING FEDERALLY SPONSORED RESEARCH

This invention was made with United States government support under Grant Number 1RO1 NSO47484 awarded by the National Institute of Neurological Disorders and Stroke. The United States government has certain rights in this invention.

### INTRODUCTION

Topographic mapping of axonal connections is a fundamental feature of nervous system wiring. In several sensory systems, such as visual, auditory and somatosensory systems, the spatial orders of the sensory receptors are smoothly and continuously mapped to brain targets. Molecular labels in the target fields are thought to specify topographic connections by activating receptors expressed in growth cones of the neurons that originate from either the sensory organs or sensory ganglia. Identification of the molecular guidance cues and their receptors involved in establishing topographic connections is critical to understanding nervous system wiring.

Neurons typically connect to multiple targets and even different brain centers. Multiple forms of axon branches are observed, including collateral and interstitial branches. During retinotectal topographic mapping, retinal ganglion cells (RGCs) project interstitial branches. These interstitial branches extend medially or laterally towards their future termination zone. Therefore, the initial direction and growth of interstitial branches influence the position of the termination zone and thus the formation of the topographic map.

It has been proposed that balanced opposing attractive and repulsive forces are necessary to generate smooth topographic maps (Prestige, 1975) (Gierer, 1983) (Fraser and Hunt, 1980) (Fraser and Perkel, 1990). However, the mechanisms by which the opposing forces are generated have yet to be fully elucidated. To date, the ephrin family of proteins, which were found to form concentration gradients within specific areas, have been identified as axon guidance cues important for map formation, particularly in the visual system.

Retinotectal projections, which are a well-characterized model system for studying map formation, are organized along the anterior-posterior and dorsal (medial)-ventral (lateral) axes. Temporal axons terminate at the anterior tectum, and nasal axons project to the posterior tectum. Ventral retinal axons connect to the medial tectum, and dorsal retinal axons find their targets at the lateral tectum. Previous studies implicated A-class ephrins in establishing anterior-posterior topographic mapping via a repulsive mechanism through the EphA receptors (Flanagan and Vanderhaeghen, 1998) (Frisen et al., 1998) (Feldheim et al., 1998) (Feldheim et al., 2000).

More recently, an attractive interaction involving ephrinB-EphB was proposed to control medial-lateral patterning of axon branch termination (Mann et al., 2002) (Hindges et al., 2002). In EphB2/EphB3 double mutant mice, axon termination zones shifted laterally, suggesting that there exists a laterally-directed EphB-independent activity that normally opposes the medially directed ephrinB/EphB activity (Hindges et al., 2002). There is a need in the art to identify molecules involved in this opposing pathway for use in modulating axonal growth.

### SUMMARY OF THE INVENTION

The present invention provides methods for modulating the medial-lateral axonal growth of a neuron. The neuron is contacted with a Wnt3 polypeptide to modulate axonal growth.

In another aspect, the present invention provides methods for modulating the medial-lateral axonal growth of a neuron in a subject in need thereof. An effective amount of a composition comprising a Wnt3 polypeptide and a pharmaceutically acceptable carrier or diluent is administered to a subject to modulate axonal growth. The present invention also provides compositions for modulating the medial-lateral axonal growth of a neuron in a subject. The compositions comprise a Wnt3 polypeptide and a pharmaceutically acceptable carrier.

In still another aspect, the present invention provides methods for modulating the medial-lateral axonal growth of a neuron by expressing an exogenous polynucleotide encoding a Wnt3 polypeptide, a Ryk polypeptide or a dominant negative Ryk that is operably connected to a promoter functional in a cell. Expression of the polypeptide in the cell modulates the axonal growth of the neuron.

In a further aspect, the present invention provides kits for modulating the medial-lateral axonal growth of a neuron. The kits comprise a Wnt3 polypeptide.

### BRIEF DESCRIPTION OF THE DRAWINGS

FIG. 1a-c are images showing in situ hybridization of Wnt3 or ephrinB1 specific probes to E10 chick tectum or to mouse P0 superior colliculus; FIG. 1d-f are graphs showing the percent maximal density of in situ hybridization of Wnt3 or ephrinB1 specific probes to E10 chick tectum or to mouse P0 superior colliculus along the medial to lateral axis; FIG. 1g is a Western blot showing Wnt3 protein in superficial optic tectum of E10 chick; FIG. 1h shows growth of E6 chick RGC explants from six different medial lateral positions as a function of Wnt3 concentration.

FIG. 2a is a diagram showing the six explants taken from retina at different dorsal-ventral positions for culture. FIG. 2b shows quantification of outgrowth of RGC axons from the six dorsal-ventral positions at different concentrations of Wnt3.

FIG. 3 shows graded expression of Ryk in chick and mouse retinal ganglion cells by in situ hybridization and signal intensity quantification. Numbers 1-6 in i-k represent six different positions along the dorsal-ventral axis of the retina (See FIG. 2a).

FIGS. 4a, e, and i show images of chick retina immunostained with anti-Ryk antibodies, FIGS. 4b, f, and j show images of DAPI stained chick retina, FIGS. 4c, g, and k show images of chick retina immunostained with anti-β-tubulin antibody; and, FIGS. 4d, h, and l show overlays of the first three images of each row.

FIG. 5 shows that Ryk is a high-affinity Wnt3 receptor (a-d). FIG. 5e-j demonstrates that Wnt3-Ryk binding is blocked by anti-Ryk antibody, but not by sFRP2 and that Wnt3-frizzled5 binding is blocked by sFRP2, but not by anti-Ryk antibody.

FIG. 6 shows that Wnt3 inhibits retinal ganglion cell axons via Ryk and stimulates retinal ganglion cell axons via Frizzled(s).

FIG. 7a is a diagram showing the electroporation method used to deliver a Wnt3 expression construct (mixed with CMV GFP at 3:1 ratio) in E7 chick tectum. Dorsal RGC axons were visualized by DiI injection to the retina at E14. FIG. 7b shows that the expression pattern of ephrinB1 in the chick tectum electroporated with Wnt3 was not altered.

FIG. 8 shows photographs of termination zone abnormalities in tectum ectopically expressing Wnt3.

FIG. 9a, left panel, is a diagram depicting the electroporation method for delivering expression constructs into the retina ganglion cells, which normally project axons to lateral tectum. FIG. 9a, right panel, a dominant-negative form of Ryk (with intracellular domain deleted) was cloned downstream of CMV promoter. FIG. 9b shows that the expression patterns of cell differentiation markers, such as ephrinB1 and EphB2, were not affected by expression of a dominant-negative form of Ryk.

FIG. 10a-b shows fluorescence photographs of chick tectum electroporated with GFP alone (a) or GFP in combination with dominant-negative Ryk (b). FIG. 10c is a diagram showing the quantification method. FIGS. 10d, e, g, and h are photographs of RGC axons showing medial-lateral interstitial branch growth in control GFP electroporated RGCs (d, g) and RGCs electroporated with dominant-negative Ryk (e, h). FIG. 10f shows quantification of medial spread and the width of the termination zone. FIG. 10i shows quantification of the ratio of medial to lateral branches in RGC axons expressing dominant-negative Ryk or eGFP control.

FIG. 11 is a diagram showing the two counterbalancing forces for medial-lateral map formation in wild-type tectum (a); dominant-negative Ryk expressing cells (b); and, in EphB2/B3−/− animals (c).

### DETAILED DESCRIPTION

Wnt3 is a secreted polypeptide that belongs to a family of proteins (Wnts) that are known to play a role in the development of a wide range of organisms. The Wnt family includes at least 18 genes that have been identified by cDNA cloning (see, e.g. WO 2004/103394). The Wnt family, including Wnt3, mediates cellular effects by binding to a family of cell surface receptors, called frizzled receptors. Wnts also bind to Ryk, which like the frizzleds, is a cell surface protein that induces a distinct cellular signaling pathway in response to Wnt binding.

As described below in the Examples, Wnt3 was discovered to exhibit continuous medial-lateral graded expression in the ventricular zone of the tectum and superior colliculus. A repulsive Wnt-Ryk pathway competes with an attractive Wnt-Frizzled interaction to affect the response to Wnt3 protein at different concentrations. The graded response may be determined by the concentration gradient of Ryk expression in the medial-lateral axis. Lateral RGCs express more Ryk, whereas medial RGCs have less Ryk. Expression of frizzled5 appears to be even along the medial-lateral axis. The net outcome of this competition is varied growth in a graded fashion along the medial-lateral axis, which, in turn, determines the topographic connections.

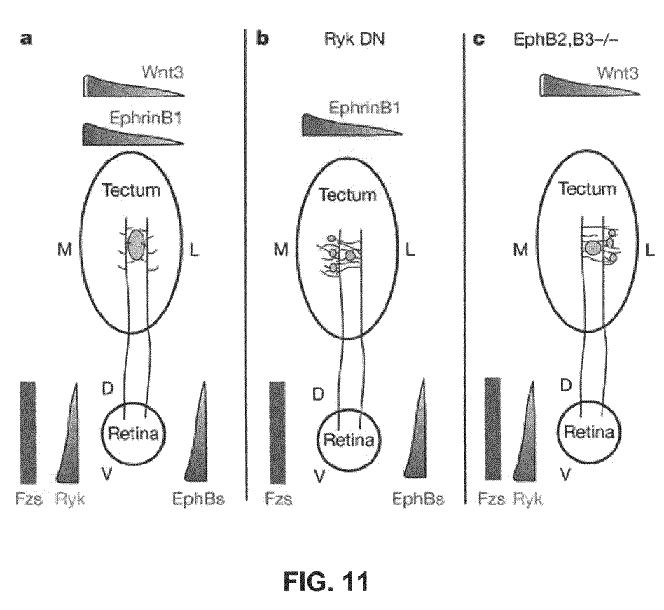

As described in the Examples, Wnt3 acts as a lateral mapping force to counterbalance the EphrinB1-EphB interaction, which has been previously described as a medial-directed mapping force (FIG. 11a). Frizzleds, which are expressed evenly along the medial-lateral axis, interact with Wnt3 to induce axonal growth. In contrast, Ryk, which is expressed at higher levels in laterally-derived cells than in medially-derived cells, interacts with Wnt3 to repel axonal growth. These two pathways compete to produce the response of the neurons to Wnt3. Blocking Wnt-Ryk function does not interfere with EphrinB1-EphB function and causes termination zones to shift medially (FIG. 11b). The termination zones shift laterally in EphB2/B3 double knockout mice (FIG. 11c). These findings suggest that the Wnt3 and EphrinB1 signaling pathways are independent of each other and act as counterbalancing forces.

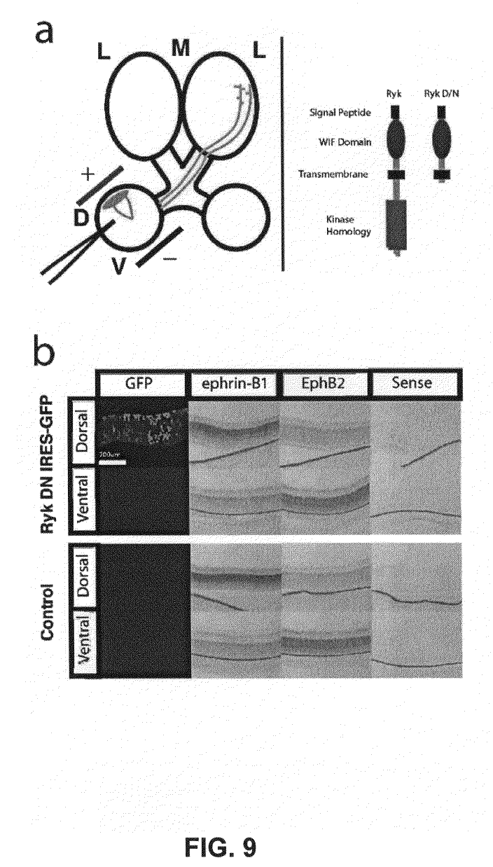

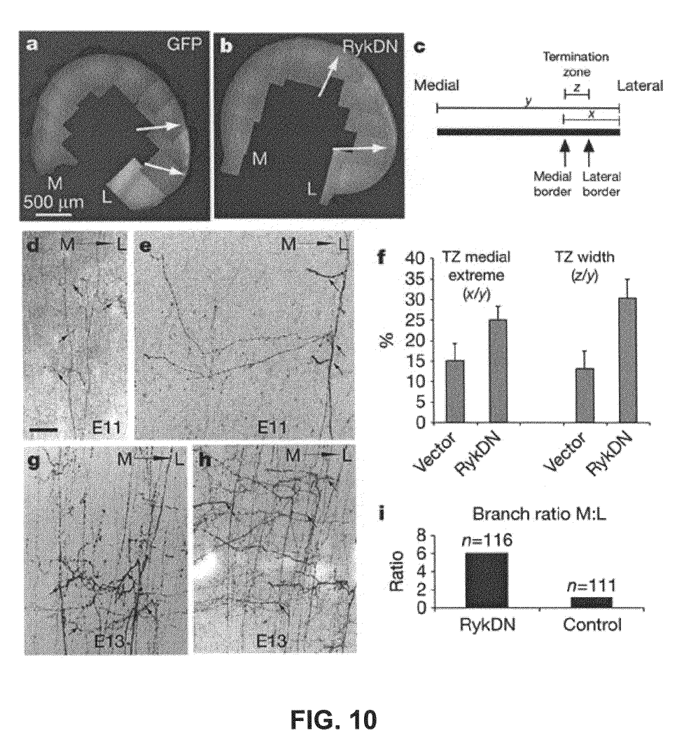

As demonstrated in the Examples, the Wnt-Ryk pathway is also required for the laterally directed interstitial branches in vivo. Blocking Wnt3 signaling using a truncated dominant negative Ryk eliminated nearly all laterally directed branches, leaving only the medially directed branches, which were found to be unusually long (see FIGS. 9a and 10 and Example 9). In contrast, in EphB2 and EphB3 double knockout mice, interstitial branches were found to be preferentially directed laterally. Therefore, Wnt3 and EphrinB1 possess opposing guidance activities for regulating interstitial branches in medial-lateral retinotectal mapping.

The present invention provides methods and compositions for modulating the axonal growth of a neuron. Modulating axonal growth includes, but is not limited to, attracting, stimulating, repressing, inhibiting or repulsing axonal growth. The growth may be limited to the axon or may include growth of the neuron as a whole and includes, but is not limited to, extension of an axon, redirection of an axon, an increase in volume of an axon or an increase in length of an axon relative to the cell body. The axonal growth can be modulated in a medial-lateral orientation relative to a specific spatial address, for example, a region of the brain.

Neurons for use in the methods may be sensory neurons or motor neurons. In one preferred embodiment, the neuron is a retinal ganglion cell. Neurons are suitably mammalian neurons. Suitably the neurons are in a brain, even more suitably the neurons are located in the superior colliculus. In one embodiment, the neuron is damaged. A neuron may be damaged by any injury or disease resulting in the loss of axonal connections including, but not limited to, traumatic injury, neurologic disease, degenerative disease, hypoxia, ischemia, anoxia and stroke.

In one embodiment, the methods comprise contacting the neuron with a Wnt3 polypeptide. In the context of the invention, contacting may be in vitro or in vivo. The Wnt3 may be in solution or provided on a solid or semi-solid support. A Wnt3 polypeptide, and any other polypeptides referred to herein, includes the full-length polypeptide or a polypeptide having at least 95% amino acid identity to the Wnt3 polypeptide. As used herein the percent amino acid identity is determined using the algorithm of Karlin and Altschul (Proc. Natl. Acad. Sci, 90:5873-5877, 1993). This algorithm is incorporated into the BLAST protein search programs, such as XBLAST and Gapped BLAST. When using these programs, the default parameters of the program are utilized. See http://www.ncbi.nlm.nih.gov.

The Wnt3 polypeptide may be provided as a concentration gradient. For example, the Wnt3 polypeptide may be provided at a relatively high concentration at one location and a relatively low concentration at a second location. The gradient may be continuous or discontinuous. For example, in RGCs a high concentration of Wnt3 is a concentration greater than or equal to 20 ng/ml and a low concentration of Wnt3 was less than or equal to 0.8 ng/ml. One of skill in the art will appreciate that the amount of Wnt3 constituting a high or low concentration will vary with the location or cell type and the level of expression of the Wnt receptors on the cells. One of skill in the art will appreciate that a gradient can be provided in a variety of ways, including, but not limited to, forming a gradient using a sustained release formulation to form a gradient by diffusion. Alternatively, the gradient may be provided on a solid or semi-solid support. The Wnt3 polypeptide can also be provided as a medial to lateral decreasing gradient in a subject, a region of the brain, ex vivo tissue sample or in vitro cultured cells, such as embryonic stem cells.

In other embodiments of the invention, the axonal growth inversely correlates with the concentration of Ryk on the neuron. When relatively low concentrations of Ryk are expressed on the neuron, such as in the medial retinal ganglion cells, axonal growth is stimulated or attracted in response to Wnt3 polypeptide. When relatively high concentrations of Ryk are expressed on the neuron, such as in the lateral retinal ganglion cells, axonal growth is repulsed or inhibited by Wnt3 polypeptide. One of skill in the art will appreciate that the concentration of Ryk on the surface of the neuron is correlated with the level of stimulation of axonal growth and that a continuum of axonal responses is contemplated.

The axonal growth of the neuron may result in the formation of an axonal connection or synapse with a second neuron, or to a muscle cell or gland. Formation of axonal connections between neurons is a critical aspect of neural system wiring and topographic map formation. In some embodiments, formation of axonal connections facilitates topographic map formation. One of skill in the art will appreciate that the methods described may be used to facilitate topographic map formation within sensory systems of the brain such as the visual system, the auditory system or the somatosensory system.

In still other embodiments, the neuron is contacted with a Wnt3 receptor inhibitor, either alone or in combination with Wnt3. Wnt3 receptors include, but are not limited to Ryk, frizzled5 and frizzled3. Wnt3 receptor inhibitors include Ryk inhibitors and frizzled inhibitors. Ryk inhibitors include, but are not limited to, an anti-Ryk antibody such as those described in the Examples that are capable blocking Wnt3-Ryk binding and a dominant negative Ryk as described in the Examples. Frizzled inhibitors include but are not limited to anti-frizzled antibodies that are capable of blocking Wnt3-frizzled binding and sFRPs. sFRPs are secreted frizzled-related proteins that can bind to Wnt proteins with high affinity and block the interaction of Wnt with frizzleds, but not with Ryk as described in the Examples. sFRPs include, but are not limited to sFRP1, sFRP2 and sFRP3.

The neuron can also be contacted with EphrinB1 polypeptide in combination with Wnt3 polypeptide. EphrinB1 polypeptide can be provided as a concentration gradient as discussed above for Wnt3 polypeptide. The Wnt3 and EphrinB1 can be provided in a single formulation or as separate formulations. The formulations can be provided substantially simultaneously, or sequentially. The concentration gradients may be in the same or opposing directions (high or low) along a given axis. One of skill in the art will appreciate that such gradients may be formed in a variety of ways.

In another embodiment, methods for modulating the medial-lateral axonal growth of a neuron in a subject are provided. The subject is suitably a mammal, and even more suitably a human. A therapeutically effective amount of Wnt3 polypeptide and a pharmaceutically acceptable carrier or diluent can be used to form a pharmaceutical composition that is administered to the subject. Administration of one or more of the pharmaceutical compositions according to this invention will be useful for regulating and for promoting neural growth or regeneration in the nervous system, for treating injuries or damage to nervous tissue or neurons, and for treating neural degeneration associated with traumas to the nervous system, neurological disorders or neurological diseases. Such traumas, diseases or disorders include, but are not limited to, optic nerve hypoplasia, aneurysms, strokes, hypoxia, anoxia, ischemia, multiple sclerosis, Alzheimer's disease, Parkinson's disease, Creutzfeldt-Jacob disease, kuru, Huntington's disease, multiple system atrophy, amyotropic lateral sclerosis (Lou Gehrig's disease), and progressive supranuclear palsy.

Determination of a preferred pharmaceutical formulation and a therapeutically effective dose regimen for a given application is within the skill of the art taking into consideration, for example, the condition and weight of the patient, the extent of desired treatment and the tolerance of the patient for the treatment.

Administration of the Wnt3 polypeptide and the other polypeptides described in this invention, include isolated and purified forms, pharmaceutically acceptable derivatives thereof, and may be accomplished using any of the conventionally accepted modes of administration of agents which are used to treat neuronal injuries or disorders.

Soluble forms of Wnt3 and the other polypeptides used herein are prepared by means well known in the art such as the Baculovirus expression system described in the Examples. The polypeptides can also be expressed by a variety of other cells types. It is anticipated that, as has been carried out for hybridoma cells that secrete antibodies (Schnell, L. and Schwab, M. E., Nature, 343, pp. 269-72 (1990); Schnell et al., Nature, 367, pp. 170-73 (1993), COS cells or other cells secreting or otherwise expressing the polypeptides described herein may be implanted into an area with damaged neurons. The cells will secrete Wnt3 polypeptide and thus modulate neuronal growth. Placement of cells expressing Wnt3 may also lead to the formation of a concentration gradient in a localized region. Optionally, cells that express Wnt3 polypeptide or other polypeptides may be encapsulated into immunoisolatory capsules or chambers and implanted into the brain or other region using available methods that are known to those of skill in the art. See, e.g., WO 89/04655; WO 92/19195; WO93/00127; EP 127,989; U.S. Pat. Nos. 4,298,002; 4,670,014; 5,487,739 and references cited therein, all of which are incorporated herein by reference.

In still other embodiments, a pump and catheter-like device may be implanted in the subject to administer the composition on a timely basis and at the desired concentration, which can be selected and empirically modified by one of skill in the art. Such pharmaceutical delivery systems are known to those of skill in the art. See, e.g., U.S. Pat. No. 4,578,057 and references cited therein, which are incorporated herein by reference.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application. Modes of administration may include oral, parenteral, subcutaneous, intravenous, intralesional or topical administration.

The Wnt3 polypeptide may, for example, be placed into sterile, isotonic formulations with or without cofactors that enhance stability. The formulation is preferably liquid, or may be lyophilized powder. The compositions also will preferably include conventional pharmaceutically acceptable carriers or diluents well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and may be administered one or more times a day.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices-include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

Liposomes containing Wnt3 can be prepared by well-known methods (See, e.g. Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). The liposomes may be of the small (about 200-800 Angstroms) unilamellar type in which the lipid content is greater than about 30 mol. % cholesterol. The proportion of cholesterol is selected to control the optimal rate of release.

The Wnt3 may also be attached to liposomes, which may optionally contain other agents to aid in targeting or administration of the compositions to the desired treatment site. Attachment of Wnt3 polypeptides to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

The present invention also provides methods for modulating the medial-lateral axonal growth of a neuron by expressing an exogenous polynucleotide encoding a Wnt3 polypeptide, a Ryk polypeptide, a dominant negative Ryk polypeptide or combinations thereof. The exogenous polynucleotide is operably connected to a promoter functional in a cell. The exogenous polynucleotides can be expressed by neurons or by other cells in the region of the neurons such that the expressed polypeptides can modulate axonal growth. Cells suitable for use include primary cells, cultured cells and cells derived from embryonic or other stem cells. The cell can be located in a subject, suitably a mammalian subject, or it can be in vitro. In another embodiment, the method comprises expressing an exogenous polynucleotide encoding a Wnt3 polypeptide, a Ryk polypeptide, or a dominant negative Ryk polypeptide and a second exogenous polynucleotide encoding a dominant negative Ryk operably connected to a promoter functional in a neuron. In yet another embodiment, the method comprises expressing an exogenous polynucleotide encoding a Wnt3 polypeptide, a Ryk polypeptide, or a dominant negative Ryk polypeptide and a second exogenous polynucleotide encoding EphrinB1 in a cell.

The polynucleotide can be introduced into the cell by electroporation, transformation, transfection, liposome delivery or any other means known in the art. The polynucleotide may be introduced into a cell in vitro, in vivo or ex vivo. If the exogenous polynucleotide is introduced into the cell in vitro or ex vivo, the cell may be implanted in vivo, as described above, after the polynucleotide is introduced into the cell. In one embodiment, a vector comprises the exogenous polynucleotide and delivers the exogenous polynucleotide to the cell. Vectors include, but are not limited to liposome and viral vectors such as an adenoviral vector, an adeno-associated viral vector, a vaccinia viral vector, a retroviral vector, a pox viral vector and a herpesviral vector.

The present invention also provides kits for modulating the medial-lateral axonal growth of a neuron. The kits comprise a Wnt3 polypeptide. Optionally, the kits may comprise instructions for carrying out the methods. The kits may also comprise EphrinB1 polypeptide, a Wnt3 receptor inhibitor, such as an anti-Ryk antibody, a dominant negative Ryk, an anti-frizzled antibody or a sFRP. Alternatively, the kits can comprise polynucleotides encoding Wnt3, EphrinB1, Ryk, or frizzleds.

All references cited herein are hereby incorporated by reference in their entireties. The following examples are meant to be illustrative only and are not intended as a limitation on the concepts and principles of the invention.

### EXAMPLES

### Example 1

### In situ hybridization of Wnt3 and EphrinB1

In situ hybridization was performed as previously described (Lyuksyutova et al., 2003). Mouse and chick Wnt3 in situ hybridization probes were isolated from E10.5 embryonic mouse brain and E6 chick brain, respectively, by RT-PCR and were used to detect the presence of Wnt3 in chick E10 tectum and mouse P0 superior colliculus.

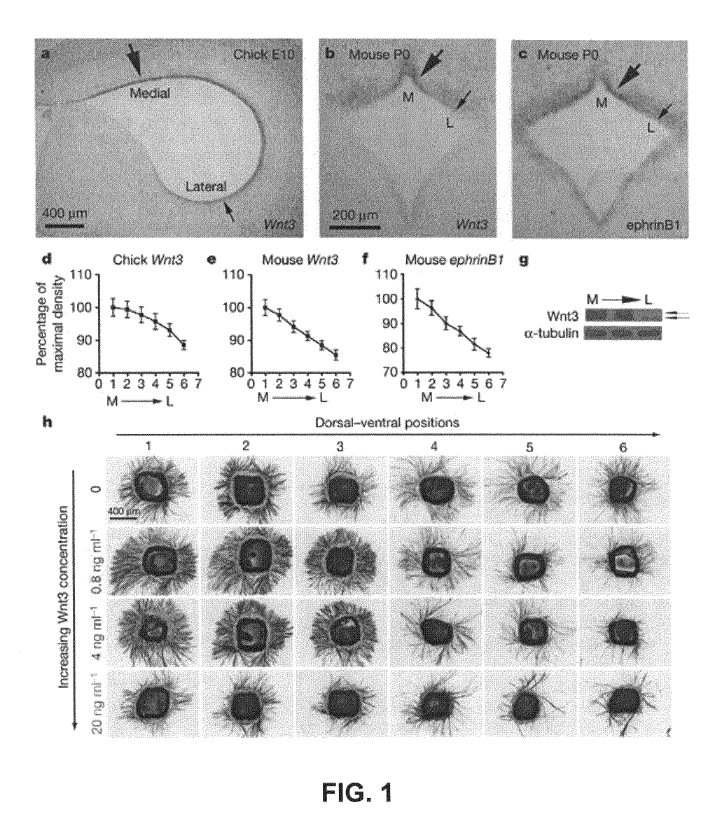

In situ hybridization showed that Wnt3 is expressed in a high-to-low gradient along the medial (dorsal)-to-lateral (ventral) axis in the ventricular zone in chick optic tectum at embryonic day 10 (E10) (FIG. 1a) and mouse superior colliculus at postnatal day 0 (P0) (FIG. 1b), similar to the expression pattern of EphrinB1 (FIG. 1c). At these stages, RGC axons have just arrived at the anterior end of the optic tectum and superior colliculus and have begun to be patterned on the pial surface.

The in situ hybridization results were quantified by measuring the signal intensity using NIH Image J. As shown in FIG. 1d-f, Wnt3 is expressed in a medial to lateral decreasing gradient similar to the expression pattern of ephrinB1. For quantification of in situ signal density, digital images of in situ hybridizations were taken and resized to 600×450 pixels. The positive grayscale signals along the dorsal-ventral axis of RGC layer of retina or the medial-lateral extent in the ventricular zone of the chick optic tectum and mouse superior colliculus were quantified with NIH Image by plot profile. The average signal density of each segment, retinal RGC layer or the ventricular zone of tectum or superior colliculus, was divided into six equal segments and calculated. Data from five sections were collected and averaged. Graphs were created using GraphPad Prism after data was normalized by defining the largest value as 100%. Owing to the nonlinear nature of the enzyme reaction, this quantification represents the direction of the in vivo gradient but may not reflect the actual steepness.

### Example 2

### Western Blot of Wnt3

Both ephrinAs and ephrinB1 transcripts are found in the ventricular zone and their proteins are thought to be transported along radial glial cells to the pial surface of the superior colliculus. To test whether Wnt3 protein is also transported to the pial surface, Western blotting was performed using an anti-Wnt3 antibody. Polyclonal anti-Wnt3 antibodies were purchased from Zymed Laboratories, Inc. and used at 0.5 μg/ml for Western blot (1:1000 dilution). Tectal tissues were isolated from the superficial layers of chick optic tectum from different medial-lateral positions and tissue lysates were equally loaded (1.5 μg protein per well) as indicated by the α-tubulin control (FIG. 1g).

Wnt3 protein was detected with a continuous medial-to-lateral decreasing gradient (M to L in FIG. 1g). Multiple bands may represent post-translational modifications. The level of Wnt3 protein at the superficial layers of medial tectum (M in FIG. 1g) is the highest and similar to that in the ventricular zone (data not shown).

### Example 3

### Biphasic and Position Dependent Responses to Wnt3

Retinal explant cultures were obtained according to a modified procedure to generate retinal ganglion cells (RGC) (Hansen et al., 2004). To determine whether Wnt3 can regulate the growth of RGC axons, their growth was examined on polycarbonated filters coated with membrane fractions of HEK293 cells expressing Wnt3, taking advantage of the fact that Wnt3 is highly hydrophobic and associates tightly with cell membranes. Wnt3-transfected HEK293 cell membranes inhibited the growth of both dorsal and ventral mouse RGC axons at higher concentrations, and stimulated the growth of medial but not lateral RGC axons at lower concentrations (not shown).

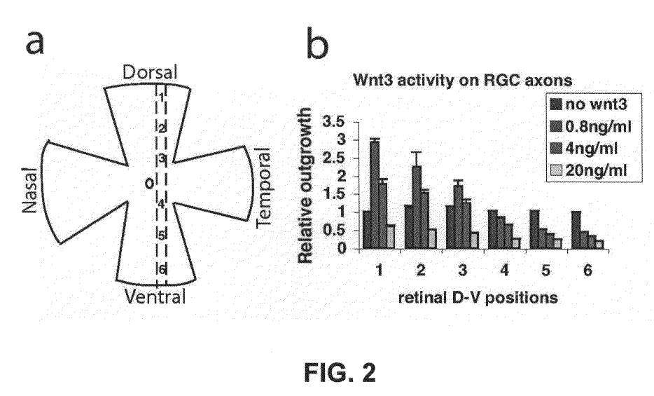

To obtain sufficient and consistent amounts of Wnt3 and obtain a quantitative measure of axonal growth, mouse Wnt3 was overexpressed in SF9 cells using the Baculovirus system. The effect of different concentrations of affinity-purified Wnt3 protein (0 ng ml⁻¹, 0.8 ng ml⁻¹, 4 ng ml⁻¹ and 20 ng ml⁻¹) on chick RGC axons from six different medial-lateral positions was evaluated (1-6 in FIG. 2a). The retinal explants were cultured on glass cover slips as described previously (Wang et al., 2002), except that tissue isolated from E6 chick retina was used.

Wnt3 protein was produced in SF9 cells, affinity purified and coated at various concentrations on the glass coverslips. Full-length mouse Wnt3 coding region was cloned into pFastBac vector (Invitrogen: Baculovirus Expression System) with a Myc tag and 6× Histidine tag at the C-terminus. This shuttle construct was used to transform DHB10 E. coli to obtain a recombinant Wnt3 Baculovirus DNA through transposition. Recombinant Wnt3 baculoviral stock was generated by transfecting SF9 insect cells with the Wnt3 baculoviral DNA. Higher titer viral stock was obtained by reamplification. SF9 cells were either infected by recombinant Wnt3 or mock viral stock at a multiplicity of infection (M. O. I.) of 0.1 viral particles/cell for 72 hours at 27° C. Cell pellets were collected and 6×His-tagged Wnt3 was purified using Ni-NTA matrices (Qiagen: Cat. No. 30210).

Explants from specific retinal positions were pooled separately and placed onto poly-D-lysine/laminin coated glass cover slips that were treated with Wnt3 or mock (control) purified protein solutions. Cover slips were incubated for 2 hours at 37° C. for each protein treatment. After 40-48 hours, explants were fixed with 4% paraformaldehyde (PFA) and subsequently immunostained with the E7 antibody against β-tubulin (Developmental Biology Hybridoma Bank). Axonal outgrowth was quantified using NIH Image; total outgrowth for each explant was normalized to the explant size in order to control for variations caused by explant size.

For each set of experiments, four explants were used in each dorsal-ventral position and Wnt3 concentration. The absolute length of outgrowth may vary slightly among different sets of experiments performed on different days. Therefore, the relative growth was quantified by normalizing the outgrowth to controls. The relative outgrowth of RGC axons for each position and Wnt3 concentration was normalized by defining the total axon length of dorsal position 1 in 0 ng/ml Wnt3 as one. Three sets of experiments were quantified, and the relative outgrowth (ratios) was averaged (FIG. 2b). Therefore, twelve explants were used for each data point. The error for dorsal position 1 and no Wnt3 is zero as it is defined as one.

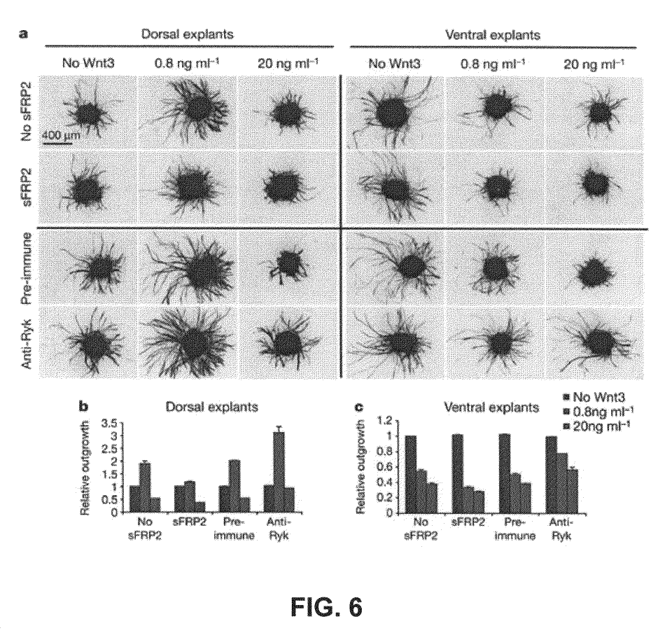

At lower Wnt3 concentrations, the growth of dorsal RGC axons was stimulated, whereas that of ventral axons was inhibited. At higher concentrations, both dorsal and ventral axon growth was inhibited (FIG. 1h). As a control, the same series of experiments were performed using control proteins from mock-infected SF9 cells and no attractive or repulsive effects were noted (data not shown). Repulsion does not appear to be due to toxicity of the Wnt3 protein preparation because Wnt3 repulsion can be blocked by antibodies against Ryk (see FIG. 6). Quantification is shown in FIG. 2b.

Retinal explants from different nasal and temporal positions were also tested. Along the nasal-temporal axis, RGC axons did not display a graded responsiveness to Wnt3, suggesting that Wnt3 does not affect anterior-posterior topographic maps (not shown). Similar experiments were also performed with mouse retinal tissues from different dorsal ventral positions in different concentrations of Wnt3, which showed similar graded responsiveness (not shown).

### Example 4

### Ryk is Expressed in a Ventral to Dorsal Decreasing Gradient in RGCs

In situ hybridization was performed as described in Example 1. Chick Ryk and Frizzled5 probe were isolated from E6 chick brain. The mouse Ryk in situ probe was cloned by RT-PCR from mouse E13.5 embryonic cDNA. The 1 kb probe included 500 nucleotides of 3′ UTR and 500 nucleotides of the coding region at the carboxyl terminus.

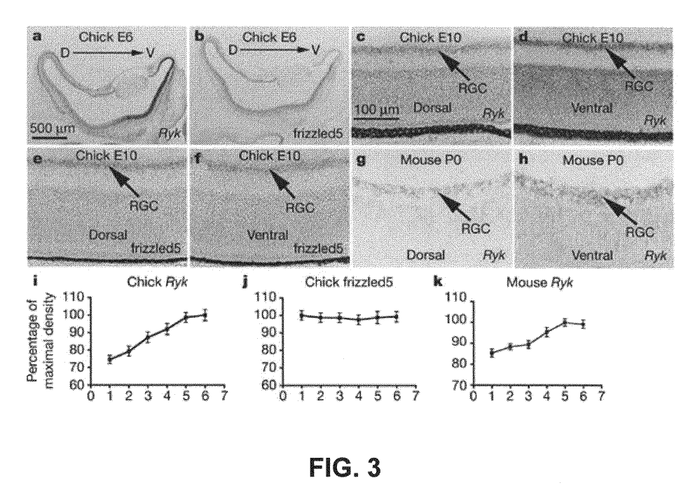

In situ hybridization showed that Ryk is expressed in a ventral-to-dorsal decreasing gradient in the RGCs of chick as early as E6 (FIG. 3a). In contrast, no dorsal-ventral gradient was observed with frizzled5 at the same stage (FIG. 3b). At a later stage (E10 chick), Ryk was also found to be expressed in a ventral-to-dorsal decreasing gradient in the RGC layer (FIG. 3c, d). No dorsal-ventral gradient of frizzled5 expression was observed (FIG. 3e, f).

In mouse P0 retina, a similar dorsal-ventral gradient of Ryk mRNA was observed (FIG. 3g, h) and no dorsal-ventral gradient of frizzled5 was detected (not shown). Because retinal tissues become much larger at E10 in chick and P0 in mouse, images are provided at a higher magnification of dorsal and ventral retina (FIG. 3c-h). Quantification of in situ measurements is shown in FIG. 3i-k. In light of the results, the differential response of retinal explants to Wnt3 is likely caused by the graded expression of the receptor Ryk.

### Example 5

### Determination of Ryk Protein Distribution on RGCs

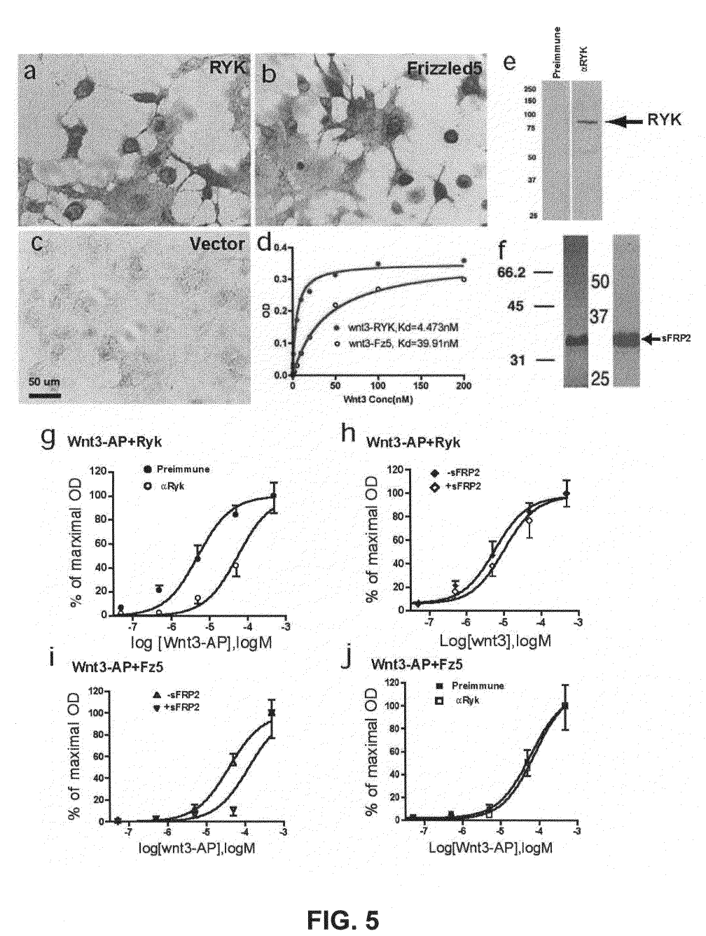

To determine the protein distribution of Ryk, antibodies against the ectodomain of Ryk were generated and immunohistochemical analysis performed. Polyclonal anti-Ryk antibodies were generated against the ectodomain of mouse Ryk, from amino acid 118 to amino acid 212, fused with maltose binding protein (in pMAL-c2X), purified, and injected into rabbits (GenBank accession number: NM013649). Specificity of the Ryk antibodies was tested by Western blotting of E11.5 mouse embryonic extracts; the antibodies recognize a highly specific band of the predicted size of 90 kD (FIG. 5e) (Hovens et al., 1992).

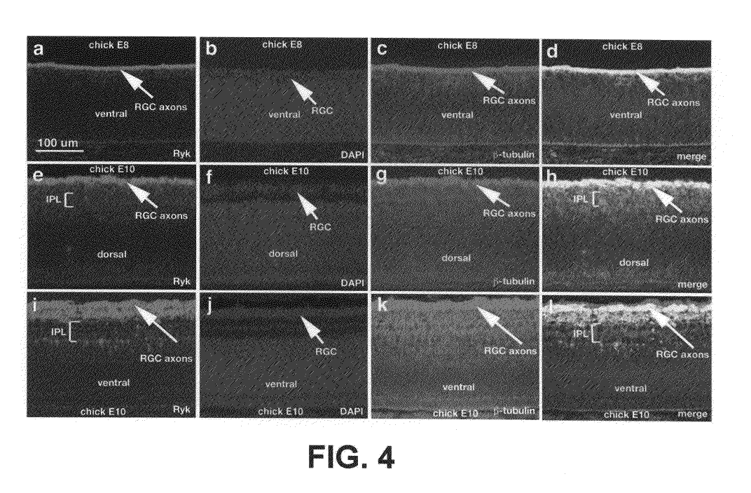

Immunostaining with chick E8 retina showed that Ryk protein is highly enriched on the axons of RGCs (FIG. 4a-d). The immunoreactive signals on the RGC axon layer in ventral retina detected by Ryk antibodies (FIG. 4a) co-localized with β-tubulin staining (E7, from Developmental Biology Hybridoma Bank; FIG. 4c), as indicated by the overlapping areas (yellow) in FIG. 4d. Very little staining in the cell body was observed at this stage (indicated by DAPI nuclear staining in FIG. 4b). Because axons from the entire dorsal-ventral axis project radially towards the optic disc to leave the eye, it is not possible to discern a potential gradient within the axon layer.

At a later stage (E10), Ryk protein continues to be enriched in axons (FIG. 4e-l, indicated by the overlapping areas with β-tubulin staining (yellow) in FIGS. 4h and l. At this stage, Ryk protein can be seen in the cell bodies of RGCs. Protein levels of Ryk on the ventral RGC cell bodies were found to be much higher than on the dorsal RGC cell bodies (FIG. 4e, i), similar to the patterns of mRNA expression (FIG. 3c, d) (arrow in FIG. 4e indicates axon layer).

### Example 6

### Ryk is a High Affinity Receptor for Wnt3

A cell-based binding assay was performed to determine whether Wnt3 can directly bind to Ryk. The assay detected the ability of a Wnt3-alkaline phosphatase (AP) fusion protein to bind to Ryk and frizzled5 expressed on cells. The protocol for the binding assay was performed as previously described (Flanagan and Leder, 1990) (Cheng and Flanagan, 1994).

Briefly, mouse Wnt3 full-length cDNA was isolated from E10.5 embryonic mouse brain by RT-PCR. Wnt3 full-length cDNA was cloned into the expression vector, pcDNA3.1 (Invitrogen). Placental alkaline phosphatase was cloned in frame into pcDNA3.1-Wnt3 to generate a Wnt3-AP fusion construct. Full-length mouse Ryk expression construct was cloned from adult mouse brain in a modified pcDNA4 His.Max vector (Invitrogen). Full-length mouse Frizzled5 cDNA was cloned from embryonic mouse tissues by RT-PCR and cloned into a modified pcDNA4 His.Max. Wnt3-alkaline phosphatase and alkaline phosphatase (control) proteins were produced by transfecting HEK293T cells and concentrated using Centriprep (Milipore). The molar concentrations of Wnt3-AP and AP fusion proteins were determined by comparing with alkaline phosphatase standards (CalBiochem).

COS cells transfected with RYK and Fz5 constructs were replated into 24 well plates 24 hours post-transfection. Wnt3-AP or AP proteins at different dilutions were incubated with COS cells for 90 min at room temperature. Cells were washed with binding buffer six times before being lysed in 1% Triton X-100 in 10 mM Tris-HCl (pH 8.0) or prepared for histochemistry. The cell lysate was centrifuged at 15,000 rpm for 2 minutes. The supernatant was heated at 65° C. for 10 minutes to inactivate endogenous phosphatase. The AP activity was quantified by measuring the OD (λ=405 nm) after incubating the lysate over an hour with 1M diethanolamine (pH 9.8), 1 mM MgCl₂ and p-nitrophenyl phosphate (Sigma). The amount of bound Wnt3-AP was quantified by subtracting the OD₄₀₅ for AP only from that of Wnt3-AP. Data were analyzed in Excel and GraphPad Prism4.

Saturating binding curves were plotted by fitting the OD₄₀₅ and Wnt3-AP concentrations with nonlinear regression, which is Y=Bmax*X/(Kd+X). Bmax is the maximal binding and Kd is the concentration of ligand required to reach half-maximal binding. In the Ryk antibody and sFRP2 blocking experiments shown in FIG. 5g-j, data were fitted with Sigmoidal dose-response equation, Y=Bottom+(Top-Bottom)/(1+10̂((LogEC50−X))). X is the logarithm of Wnt3-AP molar concentration. Y is the normalized O.D. by defining the largest value as 100%. The best-fit value of LogEC50 between data sets was compared with F test.

Myc- and 6×His-tagged sFRP2 protein was over expressed in SF9 cells with the Baculovirus system as described above and affinity purified. The purified sFRP2 protein was verified by SDS-PAGE and a single band of predicted size was detected by silver staining (˜33 kd) (left panel in FIG. 5f) and confirmed with Western blot by anti-Myc antibody (right panel in FIG. 5f).

As shown in FIG. 5a-c, Wnt3-AP does bind to Ryk as well as Frizzled5. To compare the relative affinity of Wnt3 for Ryk and the Frizzleds, the binding of the Wnt3-AP fusion protein to Ryk and frizzled5 was quantified using the cell-based binding assay. Ryk is a high-affinity receptor for Wnt3 with a K_{d} of 4.473 nM, and the K_{d} for the Wnt3-Frizzled5 interaction is 39.91 nM (FIG. 5d). Frizzled3 has similar affinity for Wnt3 as Frizzled5 (not shown). Therefore, Ryk is a higher-affinity receptor for Wnt3 than are Frizzled5 and Frizzled3.

In addition, anti-Ryk antibodies (50 μg ml⁻¹) can specifically block Wnt3-Ryk binding (FIG. 5g, P<0.0001) but not Wnt3-Frizzled5 binding (FIG. 5j, P=0.1069). In contrast, sFRP2 (0.2 μg ml⁻¹) can block Wnt-Frizzled5 binding (FIG. 5i, P=0.0045) but cannot block Wnt-Ryk binding (FIG. 5h, P=0.1094). Similar results were obtained for Frizzled3 (not shown). The mechanism of Wnt-Ryk binding may be different from that of Wnt-Frizzled binding. The Wnt-binding domain in the Frizzled protein is the cysteine rich domain (CRD) and the domain in Ryk for Wnt binding is the structurally unrelated Wnt-inhibitory factor (WIF) domain. Because of the differential blocking effect, sFRP2 protein and anti-Ryk antibodies can be used to discern the function of Frizzled and Ryk, by specifically blocking the binding of Wnt3 to Frizzled(s) or Ryk, respectively.

### Example 7

### Ryk Mediates Inhibition and Frizzleds Mediate Stimulation

To address the role of Ryk on RGC axons, anti-Ryk antibodies were evaluated for the ability to block the Wnt3 effects on dorsal and ventral axons at low and high concentrations (0.8 ng ml⁻¹ and 20 ng ml⁻¹) (FIG. 6). Retinal explants were obtained and experiments were performed similar to those described in Example 3 above. For dorsal retinal explants, retinal tissue from position 2, as indicated in FIG. 2a, was dissected ad cultured. For ventral explants, explants from position 5, as shown in FIG. 2a, were used. Ryk antibodies, sFRP2 or pre-immune sera were added to the culture medium. Quantification methods were the same as in FIG. 2b, except the relative outgrowth was normalized to no sFRP2 and no Wnt3 for the explants. Therefore, the n for each data point is 12. The errors for no sFRP2 for both dorsal and ventral explants were zero as they were defined as one. Inhibition of ventral axons by Wnt3 at both concentrations, and dorsal axons at 20 ng ml⁻¹, can be blocked by the Ryk antibodies (50 μg ml⁻¹). However, Wnt3-mediated stimulation of dorsal axons at 0.8 ng ml⁻¹ cannot be blocked by the anti-Ryk antibodies (FIG. 6a).

Several lines of evidence suggest that the Ryk antibodies are highly specific. The specificity of the Ryk antibodies was first tested by Western blot (FIG. 5e). In binding assays, the Ryk antibodies only blocked the binding of Wnt3 to Ryk (FIG. 5g) but not the binding to Frizzled5, suggesting that the Ryk antibodies do not cross-react with Frizzled5 (FIG. 5j) or Frizzled3 (not shown). In addition, the Ryk antibodies did not block the stimulation of dorsal RGC axons by low concentrations of Wnt3 (FIG. 6a). This result itself also serves as an internal control for the specificity of the antibodies. The anti-Ryk antibodies were previously described (see Liu et al., 2005, FIG. 4c-f).

Neither preimmune nor anti-Ryk postimmune sera had any effect on the growth of RGC axons in the absence of Wnt3 (FIG. 6b, c), suggesting that the rabbit sera did not promote growth in general. It is interesting that the Ryk antibodies allowed for Wnt3-mediated growth promotion (compare pre-immune and anti-Ryk in FIGS. 6b and c). When repulsion is inhibited by the Ryk antibodies, the attractive pathway takes over and shifts the balance. It should be noted that although anti-Ryk antibodies completely blocked the inhibition of dorsal RGC axons at a higher Wnt3 concentration (20 ng ml⁻¹) (compare preimmune and anti-Ryk in FIG. 6b), they did not completely block Wnt3 inhibition of ventral axons, particularly at a high concentration (up to 80% for 0.8 ng ml⁻¹ and up to 55% for 20 ng ml⁻¹) (compare preimmune and anti-Ryk in FIG. 6c). To address whether Frizzled(s) mediate stimulation or inhibition by Wnt3, the purified sFRP2 protein was tested at two concentrations of Wnt3. The stimulation of dorsal explants at low concentrations of Wnt3 (0.8 ng ml⁻¹) can be blocked by sFRP2 (0.2 μg ml⁻¹), whereas the inhibition of ventral and dorsal axons at a higher Wnt3 concentration (20 ng ml⁻¹) cannot be blocked by sFRP2 (FIG. 6a).

### Example 8

### Ectopic Wnt3 Expression in Tectum Repelled the Termination Zones

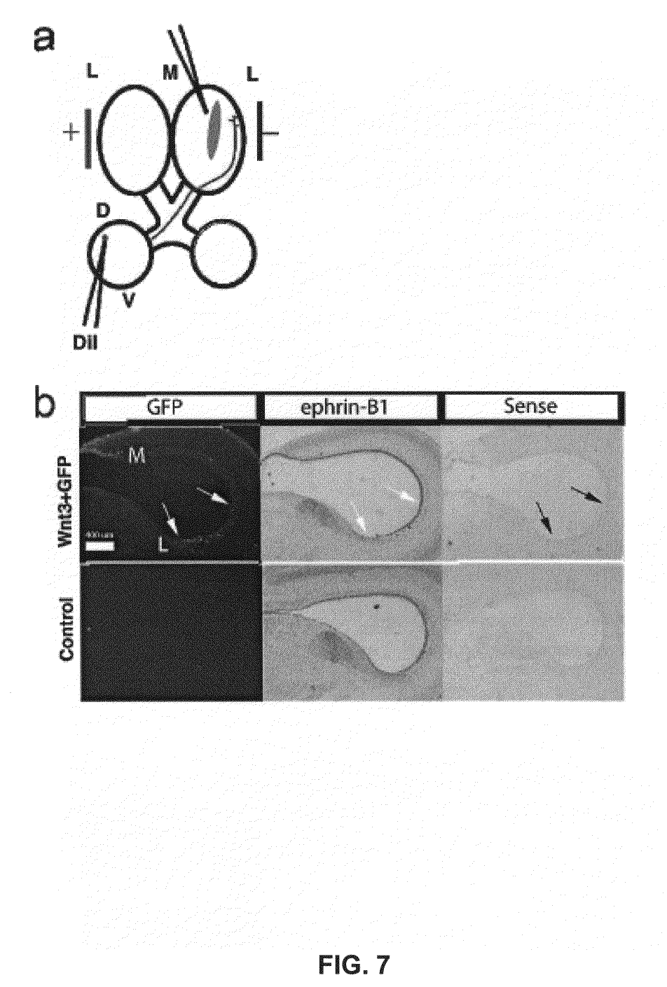

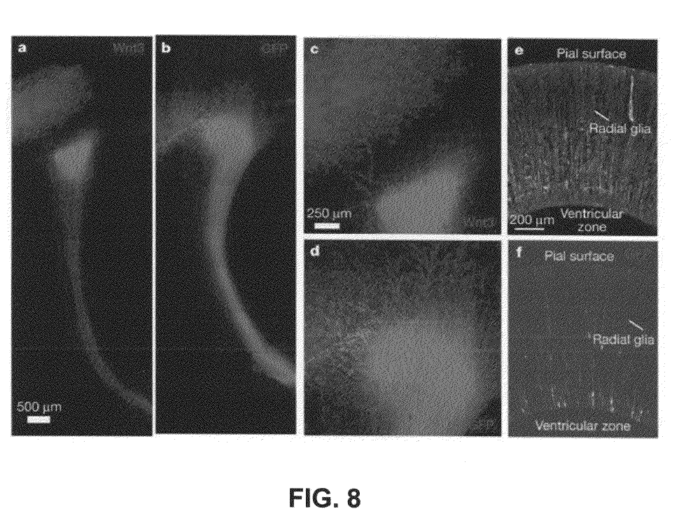

To test whether Wnt3 repels ventral axon termination zones in vivo, Wnt3 was overexpressed by electroporating vector DNA encoding Wnt3 in the ventricular zone of chick optic tectum. E7 chick tecta were electroporated with a CMV-Wnt3 expression construct mixed with a CMV-enhanced GFP (eGFP) construct (Wnt3:eGFP=3:1) to visualize the electroporated area, and at E13 RGC axons were labeled with a focal injection of DiI (diagram in FIG. 7a). Tecta were then harvested at E14 for analyses of retinotectal projections (FIG. 8). Because the tectum is patterned by E3, expressing Wnt3 four days later (E7) should not alter the patterning of tectum. In addition, the expression pattern of ephrinB1 in the chick tectum electroporated with Wnt3 was tested. The normal medial-lateral gradient of ephrinB1 was not altered (FIG. 7b).

Although EphrinAs and EphrinB1 are expressed in the ventricular zone, it has been proposed that the ephrins can be transported along radial glial to reach the pial surface to regulate RGC axon targeting (Drescher et al., 1995) (Hindges et al., 2002). Wnt3 protein can be detected in the superficial layers of the chick optic tectum, although Wnt3 mRNA was found expressed in the ventricular zone, suggesting that Wnt3 is transported to the pial surface along radial glial fibers similar to the Ephrins.

The RGC axon termination zone labeled with DiI, and was repelled by ectopic Wnt3 (FIG. 8a, c, n=7) as compared to the green fluorescent protein (GFP) only (FIG. 8b, d, n=6), confirming a repulsive role of Wnt3 in guiding the termination of RGC axons. Immunostaining with the radial glial marker, HS, confirmed that radial glial fibres are present in chick tectum at this stage (FIG. 8e). A slice of chick optic tectum electroporated with a GFP construct in the ventricular zone was stained with a GFP antibody. Many radial glial cells were found to express GFP. GFP was also detected on the pial surface, confirming that proteins expressed in the radial glial cells in the ventricular zone introduced by electroporation can be transported to the pial surface (FIG. 8f).

### Example 9

### Dominant-Negative Ryk Caused a Medial Shift of the Termination Zone

We predicted that by blocking Wnt3-Ryk function, the termination zone should shift medially. The conventional knockout mouse approach was not useful because Wnt3 and Ryk knockout mice die and cannot be used to evaluate the function of Wnt3-Ryk in mice as termination zones form at postnatal day 8. To circumvent this difficulty, a dominant-negative form of Ryk was generated and expressed in chick RGCs in the dorsal aspect of retina by in ova electroporation at E5 (FIG. 9a, right panel).

The truncated Ryk construct, with intracellular domain deleted, was cloned into pcDNA3 and pCIG2 (CMV-enhanced β-actin promoter with IRES GFP marker), a gift from Franck Polleux. The truncated Ryk protein only contains Ryk ectodomain and the transmembrane domain, missing the intracellular domain. The intracellular domain was shown to be required for axon guidance in the fly homologue, Derailed. The expression patterns of cell differentiation markers, such as EphrinB1 and EphB2, were examined and these markers normal graded expression patterns were not affected by expression of dominant negative Ryk (FIG. 9b).

To visualize RGC axons, a mixture of the dominant-negative Ryk and a cytomegalovirus (CMV)-GFP construct at a 3:1 ratio (Ryk DN:GFP) were co-electroporated. Mixing these two constructs allows us to determine the width of the termination zone (because some of the RGC axons will express the GFP control only) and the relative medial-lateral position of the termination zone when the Ryk dominant-negative construct is expressed. The Ryk dominant-negative construct was expressed in the dorsal retina. The dorsal axons normally target the lateral optic tectum, allowing testing of whether a Wnt-Ryk interaction mediates lateral-directed axon termination.

On E14, contralateral tecta were harvested, and sectioned (250-300 μm thick) perpendicular to the anterior-posterior axis with a vibratome. Sections were photographed on an epifluorescence microscope (FIG. 10a, b). Results were quantified from four Ryk dominant-negative and three control experiments (FIG. 10c, f). The relative medial-lateral positions of termination zones are dependent on the dorsal-ventral position of electroporation in the retina. Therefore, the results of the medial extreme (the most medial border) of the termination zone have higher system error. However, the results on the width of termination zones are independent of the dorsal-ventral positions of electroporation and therefore are less prone to variations caused by the site of electroporation.

RGC axons, co-electroporated with the dominant-negative Ryk construct, formed wide termination zones that extended more medially (FIG. 10b) compared to GFP control (FIG. 10a). These termination zones typically expand to at least twice the normal size, and the medial extreme of the termination zone extended widely towards the dorsal midline and only shifted medially. Results were quantified from four Ryk dominant-negative and three control experiments (FIG. 10f). Dorsal RGC axons terminate at the lateral edge of the tectum. The results depicted in FIG. 10f were calculated by the following method which is also diagrammed in FIG. 10c. Termination zone (z) is the area with eGFP signal observed in the vibratome section. The TZ width is defined as the ratio of the length of the termination zone (z) over the entire length of the tectum along the medial-lateral axis (y). The TZ medial extreme is defined as the ratio of the distance from the lateral edge to the medial border of the termination zone (x) over the distance of the entire medial-lateral axis (y). The termination zone shifted medially and expanded in size when dominant-negative Ryk was expressed in the dorsal retinal ganglion cells.

The relative medial-lateral positions of the termination zone are dependent on the dorsal-ventral position of electroporation in the retina. Therefore, the results of medial extreme of the termination zone have a higher system error. However, the results on the width of the termination zones are independent of the dorsal-ventral positions of electroporation and therefore are less prone to error caused by the site of electroporation. Taken together, these results suggest that Ryk-mediated Wnt3 repulsion is required for normal termination zone formation in vivo and normally drives axon terminals laterally until they reach a low enough concentration of Wnt3, which stabilizes the termination zones as the assays showed that dorsal axons were attracted by low levels of Wnt3 (FIG. 1h).

### Example 10

### Dominant-Negative Ryk Eliminated Lateral-Directed Interstitial Branches

To further characterize the topographic map shift and analyse interstitial branches, a construct with CMV-enhanced chick β-actin promoter driving dominant-negative Ryk, followed by internal ribosomal entry site (IRES)-eGFP was created. Using this construct, all green axons should express the dominant-negative Ryk, allowing details of branch formation from the primary RGC axons shafts to be observed. This construct was introduced into RGC cells at E7 by electroporation, and the axon projections in the whole mount tectum were analysed from E11 to E13 (FIG. 10d-h). Tecta were flat mounted on glass slides and photographed with a confocal microscope (FIG. 10d, e, g, h). We found that in Ryk dominant negative-expressing axons, very few lateral-directed branches were observed at the termination zone and only medial-directed branches were present (FIG. 10e, h) and they were typically longer than in GFP control-electroporated axons, which displayed almost equal length and frequency of interstitial branches of both directions (FIG. 10d, g). Results were quantified by counting 116 branches in 27 tecta for the Ryk dominant-negative construct and 111 branches in 20 tecta for the GFP only control (FIG. 10i). These long medial branches eventually formed multiple smaller termination zones medial to their appropriate positions along the medial-lateral axis of the tectum (not shown).

### Example 11

### Guidance of Medial-Lateral Topographic Map Formation of a Subject

Wnt3 acts as a lateral mapping force in the optic tectum to counterbalance the EphrinB1-EphB interaction, which is a medial-directed mapping force (FIG. 11a). The Wnt3 and EphrinB1 signaling pathways are probably independent of each other, as blocking of Wnt-Ryk function allows axons to still respond to the EphrinB1-EphB function, causing termination zones to shift medially (FIG. 11b), and the termination zones to shift laterally in EphB2/B3 double knockout mice (FIG. 11c). This information will be applied to repair damaged neuronal connections. After experimentally inducing neuronal damage in a mouse model, the interstitial axonal connections will be reestablished and the topographic map restored.

The topographic map will be restored by generating Wnt3 and/or EphrinB1 decreasing medial to lateral concentration gradients as depicted in FIG. 11. The Wnt3 or EphrinB1 will be provided either by administration of a Wnt3 or EphrinB1 polypeptide containing pharmaceutical composition or by expressing Wnt3 or EphrinB1 polynucleotide in cells. The topographic map restoration may be enhanced by localized treatment with a Wnt3 or EphrinB1 receptor inhibitor, such as a dominant negative Ryk, an anti-Ryk antibody, an anti-EphB antibody, or a sFRP. Treatment with a therapeutically effective amount of the disclosed polypeptides will result in interstitial axonal growth and topographic map formation.

### REFERENCES

Cheng, H. J., and Flanagan, J. G. (1994). Identification and cloning of ELF-I, a developmentally expressed ligand for the Mek4 and Sek receptor tyrosine kinases. Cell 79, 157-168.
Dann, C. E., Hsieh, J. C., Rattner, A., Sharma, D., Nathans, J., and Leahy, D. J. (2001). Insights into Wnt binding and signaling from the structures of two Frizzled cysteine-rich domains. Nature 412, 86-90.
Drescher, U., Kremoser, C., Handwerker, C., Loschinger, J., Noda, M., and Bonhoeffer, F. (1995). In vitro guidance of retinal ganglion cell axons by RAGS, a 25 kDa tectal protein related to ligands for Eph receptor tyrosine kinases. Cell 82, 359-370.
Feldheim, D. A., Kim, Y. I., Bergemann, A. D., Frisen, J., Barbacid, M., and Flanagan, J. G. (2000). Genetic analysis of ephrin-A2 and ephrin-A5 shows their requirement in multiple aspects of retinocollicular mapping. Neuron 25, 563-574.
Feldheim, D. A., Vanderhaeghen, P., Hansen, M. J., Frisen, J., Lu, Q., Barbacid, M., and Flanagan, J. G. (1998). Topographic guidance labels in a sensory projection to the forebrain. Neuron 21, 1303-1313.
Flanagan, J. G., and Leder, P. (1990). The kit ligand: a cell surface molecule altered in steel mutant fibroblasts. Cell 63, 185-194.
Flanagan, J. G., and Vanderhaeghen, P. (1998). The ephrins and Eph receptors in neural development. Annu Rev Neurosci 21, 309-345.
Fraser, S. E., and Hunt, R. K. (1980). Retinotectal specificity: models and experiments in search of a mapping function. Annu Rev Neurosci 3, 319-352.
Fraser, S. E., and Perkel, D. H. (1990). Competitive and positional cues in the patterning of nerve connections. J Neurobiol 21, 51-72.
Frisen, J., Yates, P. A., McLaughlin, T., Friedman, G. C., O'Leary, D. D., and Barbacid, M. (1998). Ephrin-A5 (AL-1/RAGS) is essential for proper retinal axon guidance and topographic mapping in the mammalian visual system. Neuron 20, 235-243.
Gierer, A. (1983). Model for the retino-tectal projection. Proc R Soc Lond B Biol Sci 218, 77-93.
Halford, M. M., Armes, J., Buchert, M., Meskenaite, V., Grail, D., Hibbs, M. L., Wilks, A. F., Farlie, P. G., Newgreen, D. F., Hovens, C. M., and Stacker, S. A. (2000). Ryk-deficient mice exhibit craniofacial defects associated with perturbed Eph receptor crosstalk. Nat Genet 25, 414-418.
Hansen, M. J., Dallal, G. E., and Flanagan, J. G. (2004). Retinal axon response to ephrin-as shows a graded, concentration-dependent transition from growth promotion to inhibition. Neuron 42, 717-730.
Hindges, R., McLaughlin, T., Genoud, N., Henkemeyer, M., and O'Leary, D. D. (2002). EphB forward signaling controls directional branch extension and arborization required for dorsal-ventral retinotopic mapping. Neuron 35, 475-487.
Hovens, C. M., Stacker, S. A., Andres, A. C., Harpur, A. G., Ziemiecki, A., and Wilks, A. F. (1992). RYK, a receptor tyrosine kinase-related molecule with unusual kinase domain motifs. Proc Natl Acad Sci USA 89, 11818-11822.
Hsieh, J. C., Rattner, A., Smallwood, P. M., and Nathans, J. (1999). Biochemical characterization of Wnt-frizzled interactions using a soluble, biologically active vertebrate Wnt protein. Proc Natl Acad Sci USA 96, 3546-3551.
Inoue, T., Oz, H. S., Wiland, D., Gharib, S., Deshpande, R., Hill, R. J., Katz, W. S., and Sternberg, P. W. (2004). C. elegans LIN-18 is a Ryk ortholog and functions in parallel to LIN-17/Frizzled in Wnt signaling. Cell 118, 795-806.
Liu, P., Wakamiya, M., Shea, M. J., Albrecht, U., Behringer, R. R., and Bradley, A. (1999). Requirement for Wnt3 in vertebrate axis formation. Nat Genet. 22, 361-365.
Liu, P., et al. (2005). Ryk-mediated Wnt repulsion regulates posterior-directed growth of corticospinal tract. Nature Neurosci. 8, 1151-1159.
Lyuksyutova, A. I., Lu, C. C, Milanesio, N., King, L. A., Guo, N., Wang, Y., Nathans, J., Tessier-Lavigne, M., and Zou, Y. (2003). Anterior-posterior guidance of commissural axons by Wnt-frizzled signaling. Science 302, 1984-1988.
Mann, F., Ray, S., Harris, W., and Holt, C. (2002). Topographic mapping in dorsoventral axis of the Xenopus retinotectal system depends on signaling through ephrin-B ligands. Neuron 35, 461-473.
McLaughlin, T., Hindges, R., and O'Leary, D. D. (2003). Regulation of axial patterning of the retina and its topographic mapping in the brain. Curr Opin Neurobiol 13, 57-69.
Mey, J., and Thanos, S. (2000). Development of the visual system of the chick. I. Cell differentiation and histogenesis. Brain Res Brain Res Rev 32, 343-379.
Patthy, L. (2000). The WIF module. Trends Biochem Sci 25, 12-13.
Prestige, M. C, and Willshaw, D. J. (1975). On a role for competition in the formation of patterned neural connexions. Proc R Soc Land B Biol Sci 190, 77-98.
Smolich, B. D., McMahon, J. A, McMahon, A. P., and Papkoff, J. (1993). Wnt family proteins are secreted and associated with the cell surface. Mol BioI Cell 4, 1267-1275.
Wang, S. W., Mu, X., Bowers, W. J., and Klein, W. H. (2002). Retinal ganglion cell differentiation in cultured mouse retinal explants. Methods 28, 448-456.

What is claimed is:

1. A method for modulating the medial-lateral axonal growth of a neuron comprising contacting the neuron with a Wnt3 polypeptide.

2. The method of claim 1, wherein the neuron is contacted with the Wnt3 polypeptide in a brain or a superior colliculus.

3. The method of claim 1, wherein the neuron is a mammalian neuron.

4. The method of claim 1, wherein the neuron is a retinal ganglion cell.

5. The method of claim 1, wherein the neuron is damaged.

6. The method of claim 1, wherein the Wnt3 polypeptide is provided as a concentration gradient.

7. The method of claim 6, wherein the gradient is provided as a medial to lateral decreasing gradient.

8. The method of claim 1, further comprising contacting the neuron with a Wnt3 receptor inhibitor.

9. The method of claim 8, wherein the Wnt3 receptor inhibitor is selected from the group consisting of a Ryk antibody, a dominant negative Ryk, a frizzled antibody, a sFRP.

10. The method of claim 1, wherein the neuron forms an axonal connection with a second neuron.

11. The method of claim 10, wherein the axonal connection facilitates topographic neural map formation.

12. The method of claim 11, wherein the topographic neural map formation occurs within a visual system, an auditory system or a somatosensory system.

13. The method of claim 1, wherein the Wnt3 polypeptide attracts the growth of the neuron.

14. The method of claim 1, wherein the Wnt3 polypeptide repels the growth of the neuron.

15. The method of claim 1, further comprising contacting the neuron with EphrinB1.

16. The method of claim 15, wherein the neuron is located in a superior colliculus and wherein the Wnt3 and the EphrinB1 are provided as a medial to lateral decreasing gradient.

17. A method for modulating the medial-lateral axonal growth of a neuron in a subject in need thereof, the method comprising administering to the subject an effective amount of a composition comprising a Wnt3 polypeptide and a pharmaceutically acceptable carrier or diluent.

18. The method of claim 17, wherein the administered composition forms a concentration gradient in the subject.

19. The method of claim 17, wherein the composition further comprises EphrinB1, a Wnt3 receptor inhibitor or a combination thereof.

20. The method of claim 17, wherein the subject is a mammal.

21. The method of claim 17, wherein the subject comprises damaged neuronal axonal connections.

22. The method of claim 17, wherein the subject has a neurologic disease.

23. A method for modulating the medial-lateral axonal growth of a neuron comprising expressing an exogenous polynucleotide encoding a Wnt3 polypeptide, a Ryk polypeptide or a dominant negative Ryk that is operably connected to a promoter functional in a cell, wherein the expression of the polypeptide in the cell modulates the axonal growth of the neuron.

24. The method of claim 23, further comprising expressing a second exogenous polynucleotide encoding a dominant negative Ryk operably connected to a promoter functional in the neuron.

25. The method of claim 23, further comprising expressing a second exogenous polynucleotide encoding EphrinB1 operably connected to a promoter functional in the cell.

26. A composition for modulating the medial-lateral axonal growth of a neuron in a subject comprising a therapeutically effective amount of Wnt3 polypeptide and a pharmaceutically acceptable carrier.

27. The composition of claim 26, wherein the pharmaceutical preparation produces a Wnt3 polypeptide concentration gradient in the subject.

28. The composition of claim 26, wherein the concentration gradient is a medial to lateral decreasing gradient.

29. The composition of claim 26, further comprising EphrinB1.

30. The composition of claim 26, further comprising a Wnt3 receptor inhibitor.

31. The composition of claim 30, wherein the Wnt3 receptor inhibitor is selected from the group consisting of a Ryk antibody, a dominant negative Ryk, a frizzled antibody, and a sFRP.

32. The composition of claim 26, wherein the subject is a mammal.

33. A kit for modulating the medial-lateral axonal growth of a neuron comprising a Wnt3 polypeptide.

34. The kit of claim 33, further comprising EphrinB1 polypeptide.

35. The kit of claim 33, further comprising a Wnt3 receptor inhibitor.

36. The kit of claim 35, wherein the Wnt3 receptor inhibitor is an anti-Ryk antibody.

37. The kit of claim 35, wherein the Wnt3 receptor inhibitor is a dominant negative Ryk.

38. The kit of claim 35, wherein the Wnt3 receptor inhibitor is a sFRP.

39. The kit of claim 38, wherein the sFRP is sFRP2.
